(19) United States
(12) Patent Application Publication
(10) Pub. No.: US 2005/0164976 A1
(43) Pub. Date: Jul. 28, 2005

(54) Combination of a cdk inhibitor and 5-fu for the tratment of cancer

(75) Inventors: Simon Richard Green, Dundee (GB); Ian Neil Fleming, Angus (GB); Rosemary Georgina Clarke, Dundee (GB); Steven James McClue, Dundee (GB)

Correspondence Address: LAHIVE & COCKFIELD, LLP., 28 STATE STREET, BOSTON, MA, 02109, US

(73) Assignee: Cyclacel Limited, Dundee (GB)

(21) Appl. No.: 10/507,883

(22) PCT Filed: Mar. 14, 2003
(86) PCT No.: PCT/GB03/01076

(30) Foreign Application Priority Data
Mar. 15, 2002 (GB) 0206203.2
Jan. 7, 2003 (GB) 0300295.3

(51) Int. Cl. A61K 31/7072; A61K 31/522; A61K 31/513

(52) U.S. Cl. 514/49; 514/269; 514/263.32

(57) ABSTRACT

A first aspect of the invention relates to a combination comprising a CDK inhibitor and 5-FU, or a prodrug thereof. A second aspect of the invention relates to a pharmaceutical product comprising a CDK inhibitor and 5-FU, or a prodrug thereof, as a combined preparation for simultaneous, sequential or separate use in therapy. A third aspect of the invention relates to a method of treating a proliferative disorder, said method comprising simultaneously, sequentially or separately administering a CDK inhibitor and 5-FU, or a prodrug thereof, to a subject.

### FIELD OF THE INVENTION

The present invention relates to a pharmaceutical combination suitable for the treatment of cancer and other proliferative disorders.

### BACKGROUND TO INVENTION

Initiation, progression, and completion of the mammalian cell cycle are regulated by various cyclin-dependent kinase (CDK) complexes, which are critical for cell growth. These complexes comprise at least a catalytic (the CDK itself) subunit and a regulatory (cyclin) subunit. Some of the more important complexes for cell cycle regulation include cyclin A (CDK1—also known as cdc2, and CDK2), cyclin B1-B3 (CDK1), cyclin C (CDK8), cyclin D1-D3 (CDK2, CDK4, CDK5, CDK6), cyclin E (CDK2), cyclins K and T (CDK9) and cyclin H (CDK7). Each of these complexes is involved in a particular phase of the cell cycle.

The activity of CDKs is regulated post-translationally, by transitory associations with other proteins, and by alterations of their intracellular localisation. Tumour development is closely associated with genetic alteration and deregulation of CDKs and their regulators, suggesting that inhibitors of CDKs may be useful anti-cancer therapeutics. Indeed, early results suggest that transformed and normal cells differ in their requirement for e.g. cyclin A/CDK2 and that it may be possible to develop novel antineoplastic agents devoid of the general host toxicity observed with conventional cytotoxic and cytostatic drugs.

The function of CDKs is to phosphorylate and thus activate or deactivate certain proteins, including, for example, retinoblastoma proteins, lamins, histone H1, and components of the mitotic spindle. The catalytic step mediated by CDKs involves a phospho-transfer reaction from ATP to the macromolecular enzyme substrate. Several groups of compounds (reviewed in N. Gray, L. Détivaud, C. Doerig, L. Meijer, Curr. Med. Chem. 1999, 6, 859) have been found to possess anti-proliferative properties by virtue of CDK-specific ATP antagonism.

Roscovitine is the compound 6-benzylamino-2-[(R)-1-ethyl-2-hydroxyethylamino]-9-isopropylpurine. Roscovitine has been demonstrated to be a potent inhibitor of cyclin dependent kinase enzymes, particularly CDK2. This compound is currently in development as an anti-cancer agent. CDK inhibitors are understood to block passage of cells from the G1/S and the G2/M phase of the cell cycle. Roscovitine has also been shown to be an inhibitor of retinoblastoma phosphorylation and therefore implicated as acting more potently on Rb positive tumors.

It well established in the art that active pharmaceutical agents can often be given in combination in order to optimise the treatment regime. The present invention therefore seeks to provide a new combination of known pharmaceutical agents that is particularly suitable for the treatment of proliferative disorders, especially cancer. More specifically, the invention centres on the surprising and unexpected effects associated with using certain pharmaceutical agents in combination.

### STATEMENT OF INVENTION

In a first aspect, the invention provides a combination comprising a CDK inhibitor and 5-FU, or a prodrug thereof.

A second aspect provides a pharmaceutical composition comprising a combination according to the invention admixed with a pharmaceutically acceptable carrier, diluent or excipient.

A third aspect relates to the use of a combination according to the invention in the preparation of a medicament for treating a proliferative disorder.

A fourth aspect relates to a pharmaceutical product comprising a CDK inhibitor and 5-FU, or a prodrug thereof, as a combined preparation for simultaneous, sequential or separate use in therapy.

A fifth aspect relates to a method of treating a proliferative disorder, said method comprising simultaneously, sequentially or separately administering a CDK inhibitor and 5-FU, or a prodrug thereof, to a subject.

A sixth aspect relates to the use of a CDK inhibitor in the preparation of a medicament for the treatment of a proliferative disorder, wherein said treatment comprises simultaneously, sequentially or separately administering a CDK inhibitor and 5-FU, or a prodrug thereof, to a subject.

A seventh aspect relates to the use of a CDK inhibitor and 5-FU, or a prodrug thereof, in the preparation of a medicament for treating a proliferative disorder.

An eighth aspect relates to the use of a CDK inhibitor in the preparation of a medicament for the treatment of a proliferative disorder, wherein said medicament is for use in combination therapy with 5-FU, or a prodrug thereof.

A ninth aspect relates to the use of 5-FU, or a prodrug thereof, in the preparation of a medicament for the treatment of a proliferative disorder, wherein said medicament is for use in combination therapy with a CDK inhibitor.

### DETAILED DESCRIPTION

The preferred embodiments as set out below are applicable to all the above-mentioned aspects of the invention.

As mentioned above, the present invention relates to a combination comprising a CDK inhibitor and 5-FU, or a prodrug thereof. 5-Fluorouracil (5-FU) is an antitumour antimetabolite, extensively used in chemotherapeutic regimes against solid tumours, particularly those of gastric or colonic origin. 5-Fluorouracil was developed in 1957 based on the observation that tumour cells utilized the base pair uracil for DNA synthesis more efficiently than normal cells of the intestinal mucosa. 5-FU is a fluorinated pyrimidine that is metabolized intracellularly to its active form, fluorodeoxyuridine monophophate (FdUMP). The active form inhibits DNA synthesis by inhibiting the normal production of thymidine. 5-FU is cell cycle phase-specific (S-phase), i.e. it blocks cell progression from the S-phase of the cell cycle.

The effect of drug combinations is inherently unpredictable and there is often a propensity for one drug to partially or completely inhibit the effects of the other. The present invention is based on the surprising observation that administering 5-FU and a CDK inhibitor (for example, roscovitine) in combination, either simultaneously, separately or sequentially, does not lead to any adverse interaction between the two agents. The unexpected absence of any such antagonistic interaction is critical for clinical applications.

Preferably, the combination has a synergistic effect, i.e. the combination is synergistic.

As mentioned above, one aspect of the invention relates to a pharmaceutical product comprising a CDK inhibitor and 5-FU, or a prodrug thereof, as a combined preparation for simultaneous, sequential or separate use in therapy.

The CDK inhibitor and 5-FU, or prodrug thereof, may be administered simultaneously, in combination, sequentially or separately (as part of a dosing regime).

As used herein, “simultaneously” is used to mean that the two agents are administered concurrently, whereas the term “in combination” is used to mean they are administered, if not simultaneously, then “sequentially” within a timeframe that they both are available to act therapeutically within the same time-frame. Thus, administration “sequentially” may permit one agent to be administered within 5 minutes, 10 minutes or a matter of hours after the other provided the circulatory half-life of the first administered agent is such that they are both concurrently present in therapeutically effective amounts. The time delay between administration of the components will vary depending on the exact nature of the components, the interaction therebetween, and their respective half-lives.

In contrast to “in combination” or “sequentially”, “separately” is used herein to mean that the gap between administering one agent and the other is significant i.e. the first administered agent may no longer be present in the bloodstream in a therapeutically effective amount when the second agent is administered.

One aspect of the present invention relates to the use of a CDK inhibitor in the preparation of a medicament for the treatment of a proliferative disorder, wherein said treatment comprises administering to a subject simultaneously, sequentially or separately 5-FU, or a prodrug thereof, and a CDK inhibitor.

Preferably, the CDK inhibitor and 5-FU, or prodrug thereof, are administered simultaneously or sequentially.

In one preferred embodiment, the 5-FU, or prodrug thereof, and CDK inhibitor are administered simultaneously.

In one particularly preferred embodiment, the CDK inhibitor is administered to the subject prior to sequentially or separately administering 5-FU, or prodrug thereof, to said subject.

Another aspect of the invention relates to a method of treating a proliferative disorder comprising the sequential administration of a therapeutically effective amount of CDK inhibitor followed by a therapeutically effective amount of 5-FU.

Another aspect of the invention relates to the use of roscovitine in the manufacture of a medicament for use in the treatment of proliferative disorders comprising the sequential administration of a therapeutically effective amount of CDK inhibitor followed by a therapeutically effective amount of 5-FU.

In an alternative preferred embodiment, 5-FU, or prodrug thereof, is administered to the subject prior to sequentially or separately administering a CDK inhibitor to said subject.

In one particularly preferred embodiment, the CDK inhibitor and 5-FU, or prodrug thereof, are administered sequentially.

In one preferred embodiment of the invention, the CDK inhibitor and 5-FU, or prodrug thereof, are each administered in a therapeutically effective amount with respect to the individual components.

In another preferred embodiment of the invention, the CDK inhibitor and 5-FU, or a prodrug thereof, are each administered in a subtherapeutic amount with respect to the individual components.

Another aspect of the invention relates to the use of a CDK inhibitor and 5-FU, or a prodrug thereof, in the preparation of a medicament for treating a proliferative disorder.

Yet another aspect of the invention relates to the use of a CDK inhibitor in the preparation of a medicament for the treatment of a proliferative disorder, wherein said medicament is for use in combination therapy with 5-FU, or a prodrug thereof.

A further aspect of the invention relates to the use of 5-FU, or a prodrug thereof, in the preparation of a medicament for the treatment of a proliferative disorder, wherein said medicament is for use in combination therapy with a CDK inhibitor.

As used herein, the term “combination therapy” refers to therapy in which the 5-FU, or prodrug thereof, and CDK inhibitor are administered, if not simultaneously, then sequentially within a timeframe that they both are available to act therapeutically within the same time-frame.

As used herein the phrase “preparation of a medicament” includes the use of the components of the invention directly as the medicament in addition to their use in any stage of the preparation of such a medicament.

The term “proliferative disorder” is used herein in a broad sense to include any disorder that requires control of the cell cycle, for example cardiovascular disorders such as restenosis and cardiomyopathy, auto-immune disorders such as glomerulonephritis and rheumatoid arthritis, dermatological disorders such as psoriasis, anti-inflammatory, anti-fungal, antiparasitic disorders such as malaria, emphysema and alopecia. In these disorders, the components of the present invention may induce apoptosis or maintain stasis within the desired cells as required.

Preferably, the proliferative disorder is a cancer or leukaemia, most preferably cancer.

In a more preferred embodiment, the proliferative disorder is a soft tissue cancer, for example, breast cancer.

In another preferred embodiment, the proliferative disorder is colorectal cancer, head or neck cancer, cervical or pancreatic cancer.

In a particularly preferred embodiment, the invention relates to the use of the combination described herein in the treatment of a CDK dependent or sensitive disorder. CDK dependent disorders are associated with an above normal level of activity of one or more CDK enzymes. Such disorders preferably associated with an abnormal level of activity of CDK2 and/or CDK4. A CDK sensitive disorder is a disorder in which an aberration in the CDK level is not the primary cause, but is downstream of the primary metabolic aberration. In such scenarios, CDK2 and/or CDK4 can be said to be part of the sensitive metabolic pathway and CDK inhibitors may therefore be active in treating such disorders. Such disorders are preferably cancer or leukaemic disorders.

Preferably, the CDK inhibitor is an inhibitor of CDK2 and/or CDK4. More preferably the CDK inhibitor is selected from roscovitine, purvalanol A, purvalanol B, olomucine and other 2,6,9-trisubstituted purines as described in WO97/20842, WO98/05335 (CV Therapeutics), WO99/07705 (Regents of the University of California).

Even more preferably the CDK inhibitor is selected from roscovitine and purvalanol A.

In one particularly preferred embodiment, the CDK inhibitor is roscovitine.

Roscovitine is the compound 2-[(1-ethyl-2-hydroxyethyl)amino]-6-benzylamine-9-isopropylpurine, also described as 2-(1-D,L-hydroxymethylpropylamino)-6-benzylamine-9-isopropylpurine. As used herein, the term “roscovitine” encompasses the resolved R and S enantiomers, mixtures thereof, and the racemate thereof.

Many anti-cancer agents are given in combination in order to optimise the treatment regime. 5-FU has been used in combination with cyclophosphamide, methotrexate (Int J Cancer (1981) 28, 91-96) and together with leucovorin in a dosing regime with cisplatin (Jpn J Clin One (2001) 31, 605-609). This latter disclosure provides summary of 5-FU combinations that have been attempted with a view to improving treatment of advance gastric cancer. Bible K C and Kaufmann S H, Cancer Res. (1997) 57: 3375-3380, described the sequential administration of flavopurinol and 5-FU. However, to date there has been no suggestion of administering 5-FU, or a prodrug thereof, in combination with roscovitine.

Even more preferably, the combination is a synergistic combination comprising roscovitine and 5-FU, or a prodrug thereof.

In a preferred embodiment, the combination of 5-FU, or a prodrug thereof, and roscovitine produces an enhanced effect as compared to either drug administered alone. The surprising nature of this observation is in contrast to that expected on the basis of the prior art.

In one particularly preferred embodiment of the invention, the prodrug is capecitabine. Thus, preferably, the combination comprises roscovitine and capecitabine.

Pharmaceutical Compositions

Although the components of the present invention (including their pharmaceutically acceptable salts, esters and pharmaceutically acceptable solvates) can be administered alone, for human therapy they will generally be administered in admixture with a pharmaceutical carrier, excipient or diluent.

A preferred embodiment of the invention therefore relates to a pharmaceutical composition comprising a CDK inhibitor and 5-FU, or a prodrug thereof, admixed with a pharmaceutically acceptable excipient, diluent or carrier.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985). Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Salts/Esters

The agents of the present invention can be present as salts or esters, in particular pharmaceutically acceptable salts or esters.

Pharmaceutically acceptable salts of the agents of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

The invention also includes where appropriate all enantiomers and tautomers of the agents. The man skilled in the art will recognise compounds that possess optical properties (one or more chiral carbon atoms) or tautomeric characteristics. The corresponding enantiomers and/or tautomers may be isolated/prepared by methods known in the art.

Stereo and Geometric Isomers

Some of the agents of the invention may exist as stereoisomers and/or geometric isomers, e.g. they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided said forms retain the appropriate functional activity (though not necessarily to the same degree).

The present invention also includes all suitable isotopic variations of the agents or pharmaceutically acceptable salts thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agents of the present invention and pharmaceutically acceptable salts thereof can generally be prepared by conventional procedures using appropriate isotopic variations of suitable reagents.

Solvates

The present invention also includes solvate forms of the agents of the present invention. The terms used in the claims encompass these forms.

Polymorphs

The invention furthermore relates to agents of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Chemical Derivatives

The invention also relates to combinations which comprise derivatives of the agents. The term “derivative” as used herein includes chemical modification of an agent. Illustrative of such chemical modifications would be replacement of hydrogen by a halo group, an alkyl group, an acyl group or an amino group.

Prodrugs

The invention further includes agents of the present invention in prodrug form. Such prodrugs are generally compounds wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include esters (for example, any of those described above), wherein the reversion may be carried out be an esterase etc. Other such systems will be well known to those skilled in the art.

In one particularly preferred embodiment, the 5-FU is in the form of a prodrug, preferably capecitabine. Capecitabine is a 5-FU prodrug which is particularly suitable for oral administration.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 2000 mg and more preferably from 50-1000 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredients can be incorporated into a cream consisting of an aqueous emulsion of polyethylene glycols or liquid paraffin. The active ingredients can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-500 mg, of active ingredient per dose.

Compositions may be formulated in unit dosage form, i.e., in the form of discrete portions containing a unit dose, or a multiple or sub-unit of a unit dose.

In a particularly preferred embodiment, the combination or pharmaceutical composition of the invention is administered intravenously.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific agents employed, the metabolic stability and length of action of that agent, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.1 to 30 mg/kg body weight, or from 2 to 20 mg/kg, more preferably from 0.1 to 1 mg/kg body weight.

As described above, each active component, the CDK inhibitor and 5-FU, or a prodrug thereof, are administered in a therapeutically effective amount preferably in the form of a pharmaceutically acceptable composition. These amounts will be familiar to those skilled in the art. By way of guidance, 5-FU is typically administered intravenously, orally or topically. Intravenous and oral doses typically comprise 250 mg or 500 mg 5-FU and are administered in accordance to a physicians direction at a total dosage depending on the weight of a patient e.g. orally at 15 mg/kg weekly, maximum dose 1 g/day, or intravenously 12 mg/kg over 4 hours, or 24-49 mg/kg over 24 hours daily for 5 days. Oral dosages are typically administered in capsules, whereas intra-venous administration is generally administered over a number of hours, typically 4 hours.

Preferably, roscovitine is administered as an orally or intravenously at a dosage of from 1 to 5 g/day. 5-FU is then administered in the manner deemed most suitable at an appropriate dosage as discussed above. Preferably, the 5-FU is administered at least 24 hours after the administration of roscovitine.

Roscovitine is typically administered orally or intravenously at a dosage of from about 0.05 to about 5 g/day, preferably from about 0.5 to about 5 g/day or 1 to about 5 g/day, and even more preferably from about 1 to about 3 g/day. Alternatively, roscovitine is preferably administered at a dosage of about 0.4 to about 3 g/day. Roscovitine is preferably administered orally in tablets or capsules. The total daily dose of roscovitine can be administered as a single dose or divided into separate dosages administered two, three or four time a day.

The present invention is further described by way of example and with reference to the following figures wherein:

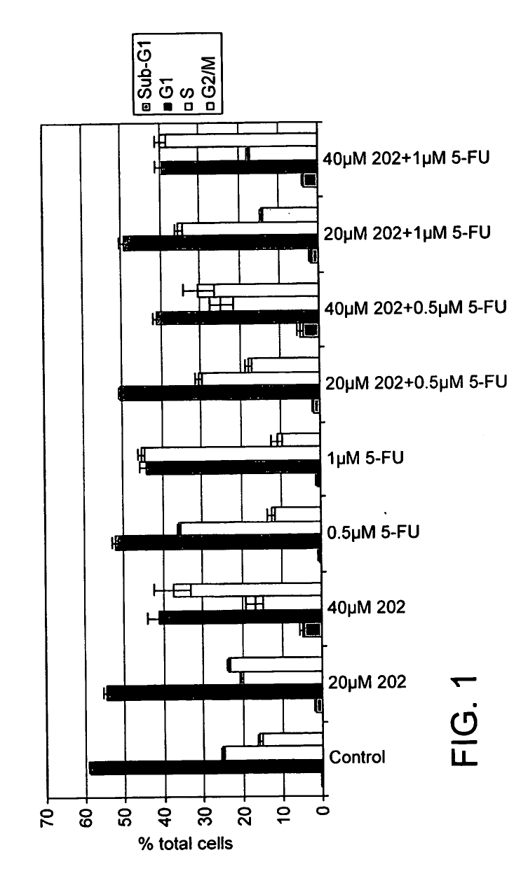

FIG. 1 shows the effect of treatment with roscovitine (referred to herein as “CYC202” or “202”), 5-FU, and [roscovitine+5-FU] on the cell cycle distribution of HT29 cells.

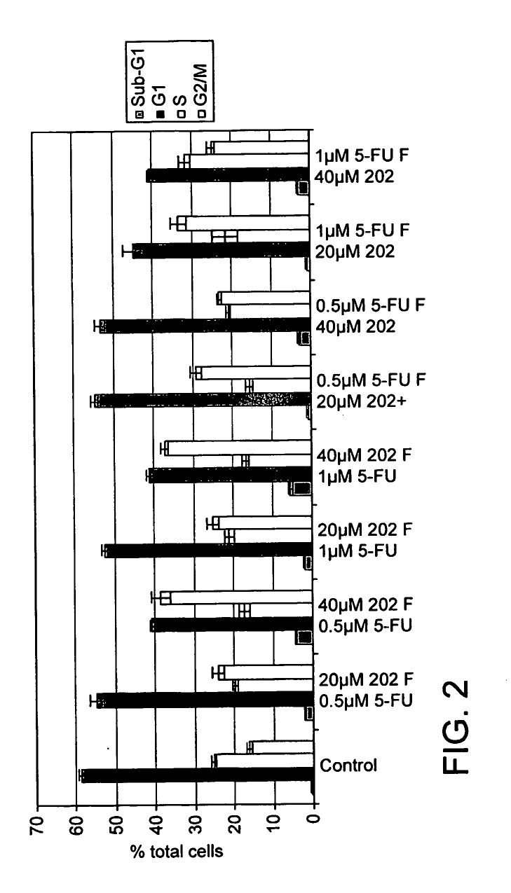

FIG. 2 shows the effect of sequential treatment with roscovitine and 5-FU on the cell cycle distribution of HT29 cells.

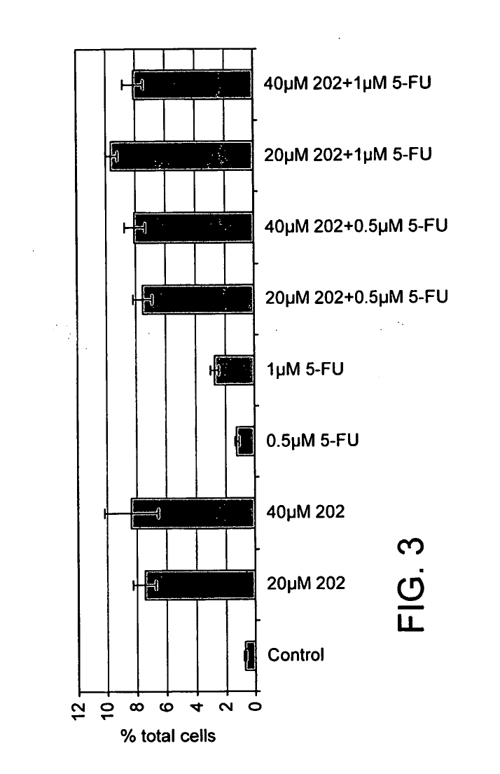

FIG. 3 shows the effect of treatment with roscovitine, 5-FU and [roscovitine+5-FU] on the activation of caspase-3 in HT29 cells.

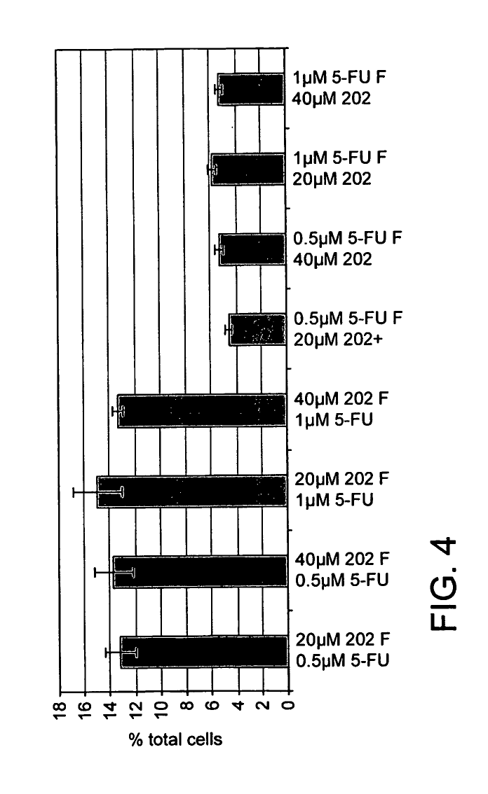

FIG. 4 shows the effect of sequential treatment with roscovitine and 5-FU on the activation of caspase-3 in HT29 cells.

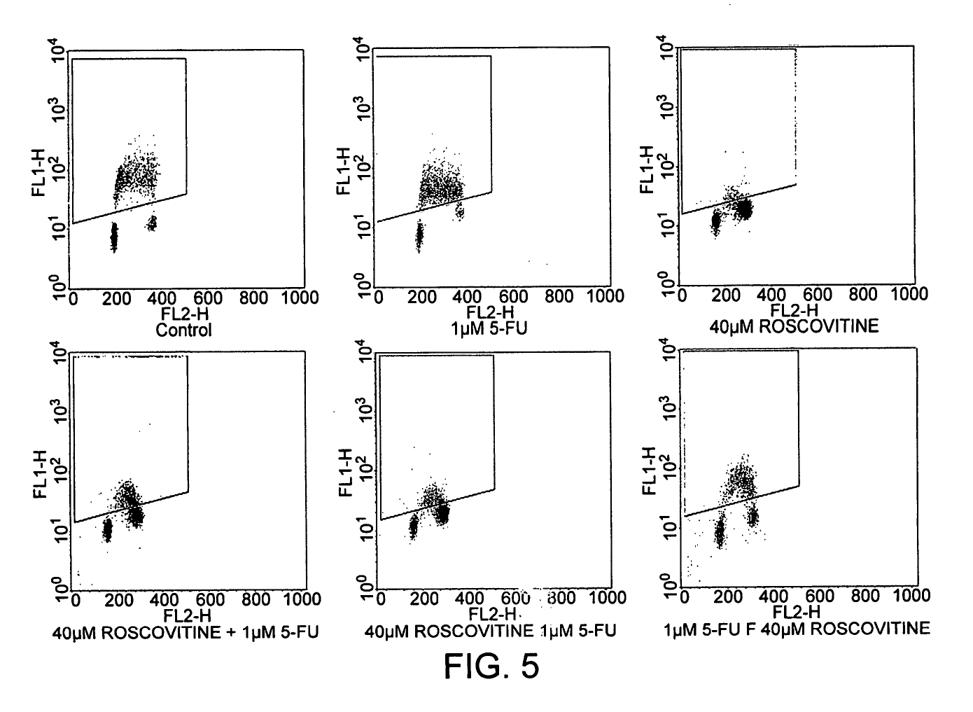

FIG. 5 shows the effect of combined treatment with roscovitine and 5-FU on the proliferation of HT29 cells, as determined by the incorporation of BrdU.

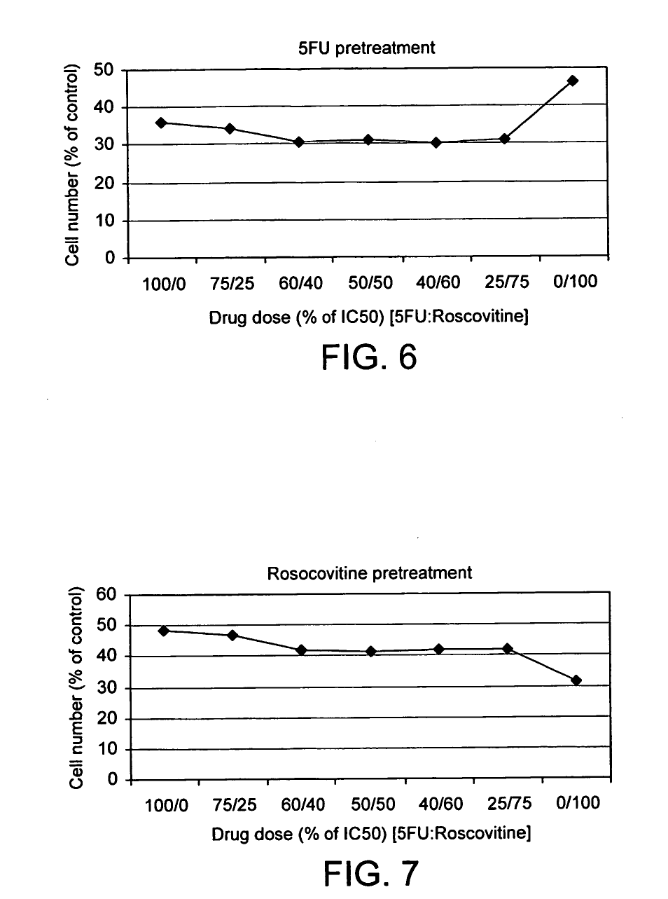

FIG. 6 shows the effect of 5-FU pretreatment on MCF7 cells; cell number (as % of control) versus drug dose (as % of IC₅₀).

FIG. 7 shows the effect of roscovitine pretreatment on MCF7 cells; cell number (as % of control) versus drug dose (as % of IC₅₀).

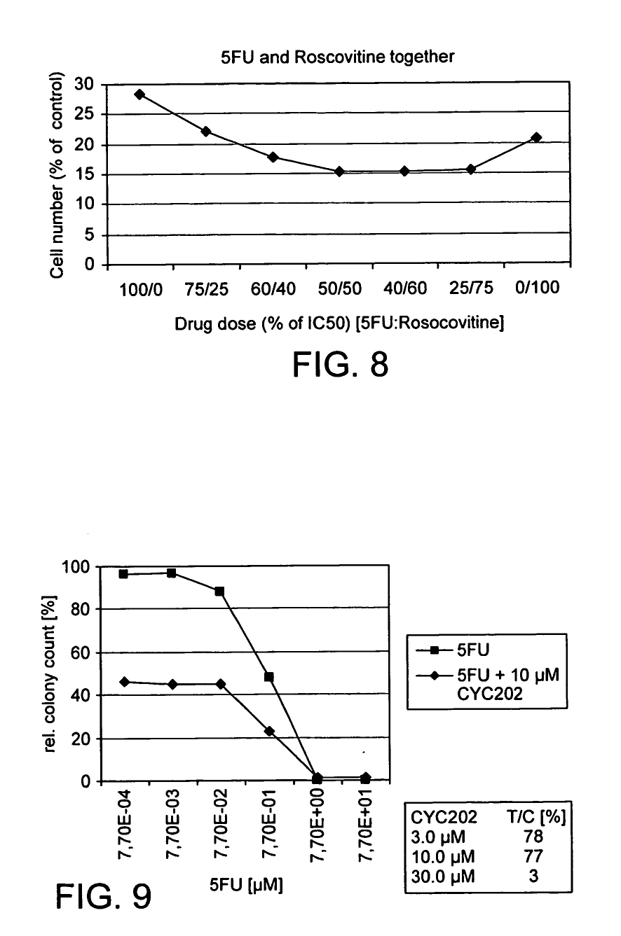

FIG. 8 shows the effect of concurrent roscovitine/5-FU treatment on MCF7 cells; cell number (as % of control) versus drug dose (as % of IC₅₀).

FIG. 9 shows the in vitro anticancer efficacy of roscovitine in combination with 5-fluorouracil (5-FU) in the human mammary cancer xenograft MAXF 857.

### EXAMPLES

Materials and Methods

HT29 human colorectal carcinoma cells were treated with roscovitine and/or 5-FU. The compounds were given singly, concomitantly and in sequence. The effects of the sequence of administration of roscovitine with 5-FU were analysed by flow cytometry and by assaying caspase-3 levels (an early marker of induction of apoptosis.)

Cell Culture

The human colorectal cancer cell line HT29 was obtained from the European Collection of Animal Cell Cultures. Cells were grown in Dulbecco's Modified Eagle's medium supplemented with 10% v/v fetal calf serum (Perbio), 100 U/ml penicillin and 100 μg/ml streptomycin. Cells were grown at 37° C., 5% v/v CO₂ in a humidified atmosphere and harvested using 0.05% w/v trypsin, 0.02% w/v EDTA. Cells were washed in media to inactivate trypsin before reseeding or analysis.

Drug Treatment

Drug treatment concentrations were selected on the basis of IC₅₀ values calculated by a cellular cytotoxicity assay. Roscovitine was dosed at either IC₅₀ (20 μM) or 2×IC₅₀ (40 μM) and 5-FU at IC₅₀ (1 μM) or 0.5×IC₅₀ (0.5 μM). For combined and sequential treatments all possible combinations of drug and concentration were evaluated.

Analysis of Cell Cycle Distribution

HT29 cells were seeded onto 90 mm diameter plates at 1×10⁶ cells per plate and incubated for 24 hours. Cells were treated with either roscovitine, 5-FU or roscovitine+5-FU at the relevant concentrations for 48 hours, except where sequential drug treatment was to be applied. In samples where sequential application of the drugs was to be evaluated, after 24 hours exposure to the first drug, media was removed from the plates to tubes and the relevant second drug added. After mixing, media was returned to the relevant plates, which were incubated for a further 24 hours. Both detached and adherent cells were then harvested. After washing once in PBS, cells were fixed in ice cold 70% v/v ethanol in water and stored at −20° C. Cells were washed twice in PBS+1% w/v BSA to remove fixative and re-suspended in PBS containing 0.1% v/v triton-X, 50 μg/ml propidium iodide and 50 μg/ml RNaseA. After incubation at room temperature for 20 minutes, cells were analysed using flow cytometry.

Activated Caspase-3 Assay

HT29 cells were seeded, treated with drug and harvested in the same way as for the determination of cell cycle distribution. Cells were fixed in 1% w/v paraformaldehyde for 30 minutes at 37° C., washed once in PBS, re-suspended in ice cold 70% v/v ethanol and stored at −20° C. before analysis. Cells were washed twice in PBS+1% w/v BSA and re-suspended in 120 μl of FITC conjugated anti-activated caspase-3 antibody (Pharmingen) diluted 1:5 in PBS+1% BSA, and incubated at room temperature for 30 minutes protected from light. After washing once on PBS+1% BSA, cells were re-suspended in PBS containing 0.1% v/v triton-X, 50 μg/ml propidium iodide and 50 μg/ml RNaseA. After incubation at room temperature protected from light for 20 minutes, samples were analysed by flow cytometry.

Assessment of Cell Proliferation by Incorporation of BrdU

HT29 cells were seeded and treated with drug in the same way as for the determination of cell cycle distribution. After 24 hours, media was replaced with that containing 10 μM BrdU and incubated for 30 minutes. Detached cells were harvested, plates were washed twice in PBS and adherent cells harvested by trypsinisation. Adherent and detached cells were pooled, washed in PBS, fixed in ice cold 70% v/v ethanol and stored at 4° C. Cells were washed twice in PBS+1% BSA and treated with 2M HCl for 20 minutes, followed by a further three washes in PBS. Cells were pelleted and 2 μl of anti-BrdU antibody (Pharmingen) added to the pellet. Cells were left at room temperature for 30 minutes and washed once in PBS+1% BSA. 50 μl of FITC conjugated anti-mouse F(ab′)₂ (Dako) diluted 1:10 in PBS+1% BSA was added to pelleted cells and samples incubated for 30 minutes at room temperature protected from light. After washing once on PBS+1% BSA, cells were re-suspended in PBS containing 0.1% v/v triton-X, 50 μg/ml propidium iodide and 50 μg/ml RNaseA. After incubation at room temperature protected from light for 20 minutes, samples were analysed by flow cytometry.

Flow Cytometry

A Becton Dickinson LSR flow cytometer was used for these studies, in accordance with the manufacturers recommendations. The argon ion laser set at 488 nm was used as an excitation source. Where used, activated caspase-3 and BrdU positive cells were designated as such on the basis of green fluorescence (530±28 nm), acquired on a logarithmic scale. Red fluorescence (575±26 nm) was acquired on a linear scale and pulse width analysis was used to exclude cell doublets and aggregates from the analysis. Cells with a DNA content of between 2n and 4n were designated as being in G1, S or G2/M phases of the cell cycle, as defined by the level of red fluorescence. Cells showing less than 2n DNA content were designated as sub-G1 cells. The number of cells in each cell cycle compartnent was expressed as a percentage of the total number of cells present.

Results

Cell Cycle Analysis

The effect of treatment with roscovitine, 5-FU and roscovitine+5-FU on the cell cycle distribution of HT29 cells is shown in FIG. 1.

Treatment with roscovitine at both 20 μM and 40 μM causes accumulation of cells in G2/M, and treatment with 0.5 μM and 1 μM 5-FU causes accumulation of cells in S phase, both in a dose dependent manner. When cells were treated concurrently with 20 μM roscovitine and either 0.5 μM or 1 μM 5-FU, accumulations in S phase were seen indicating that, at both concentrations, 5-FU can prevent the G2/M block of 20 μM roscovitine. However, when the level of roscovitine was increased to 40 μM, accumulations in G2/M were seen at both 0.5 μM and 1 μM 5-FU, indicating that by increasing the level of roscovitine, the effect of 5-FU can be overcome.

The effect of sequential treatment with roscovitine and 5-FU on the cell cycle distribution of HT29 cells is shown in FIG. 2.

When roscovitine is dosed prior to 5-FU, the cell cycle distributions are much the same as those obtained for roscovitine alone. Increasing the concentration of 5-FU from 0.5 μM to 1 μM had no obvious effect on cell cycle distribution. When 5-FU was dosed prior to roscovitine the accumulations in G2/M, usually seen on treatment with roscovitine were not as marked, particularly in those samples treated with 40 μM roscovitine. In samples treated with 1 μM 5-FU followed by 40 μM roscovitine there was a marked increase in S phase cells. These results suggest that 5-FU is capable of blocking the cell cycle effect of roscovitine.

Caspase Activation

FIG. 3 shows the effect of treatment with roscovitine, 5-FU and (roscovitine+5-FU) on the activation of caspase-3 in HT29 cells.

Roscovitine induces activation of caspase-3 (an early marker of apoptotic cell death) in a dose dependent manner, as does 5-FU, albeit to a lesser extent. When dosed in combination the results are similar to those seen with roscovitine alone, indicating that concurrent treatment with the two drugs at the levels tested does not increase cell death (ie. there is no evidence of an additive or synergistic effect on the basis of this particular assay).

The effect of sequential treatment with roscovitine and 5-FU on the activation of caspase-3 in HT29 cells is shown in FIG. 4.

Treatment with roscovitine prior to 5-FU showed high levels of caspase-3 activation (cell death), with similar levels being seen in all drug concentration combinations. More specifically, the administration of roscovitine prior to 5-FU results in an improved activation of caspase-3, and this activation is greater than that achieved by 5-FU or roscovitine alone. Thus, on the basis of this assay the sequential administration of a CDK inhibitor, such as roscovitine, followed by 5-FU produces a maximal effect on the induction of apoptosis as compared to either drug administered alone. Without wishing to be bound by theory, it is believed that sequential treatment with roscovitine followed by 5-FU prevents the 5-FU-induced block in the S-phase.

When 5-FU was dosed prior to roscovitine, the levels of cell death were greatly reduced suggesting that 5-FU may block the induction of cell death by roscovitine, when measured by this particular assay.

BrdU Analysis

Treatment with 40 μM roscovitine results in reduced proliferation as shown by the reduction in the number of cells incorporating BrdU (3.8%). Treatment with 1 μM 5-FU, increased the number of cells incorporating BrdU to 64.3% but the intensity of BrdU labelling was lower than that seen in the control, which is indicative of a drug which causes arrest in S phase. When dosed concurrently, 40 μM roscovitine+1 μM 5-FU resulted in more cells incorporating BrdU than roscovitine alone. This indicates that the presence of 5-FU is blocking the effect of roscovitine, as suggested by the cell cycle distribution results. Sequential treatment with 40 μM roscovitine prior to 1 μM 5-FU gave results similar to those for 40 μM roscovitine alone. Sequential treatment with 1 μM 5-FU followed by 40 μM roscovitine showed a reduction in the proliferation compared to 5-FU alone and an increase compared to roscovitine alone, indicating that dosing 5-FU prior to roscovitine may be inhibiting the effect of roscovitine.

The effect of combined treatment with roscovitine and 5-FU on the proliferation of HT29 cells, as determined by the incorporation of BrdU is shown in FIG. 5.

Roscovitine and 5FU Treatment of MCF7 Cells

MCF7 cells were seeded at 3,000/well in 96 well plates. Cell were allowed to settle overnight, and medium replaced with drug-containing medium the next day. Nine different drug treatments were tested, in triplicate, in each plate. The IC₅₀ values for the two drugs of interest in MCF7 cells was 8 μM (5-FU) and 10 μM roscovitine. The experiment model ranged from treating cells with 100% of the IC₅₀ for 5-FU (treatment 1), to 100% of the IC₅₀ for roscovitine (treatment 7). Treatments 2-6 involved treating cells with a range of different ratios of 5-FU to roscovitine, as shown in Table 1 below. Two additional controls were also run, which involved treatment with no drug (treatment 8) or with 100% IC₅₀ of both drugs (treatment 9).

TABLE 1

| | Treatment | | | | | | | | |
|---|---|---|---|---|---|---|---|---|---|
| Drug | 1 | 2 | 3 | 4 | 5 | 6 | 7 | 8 | 9 |
| 5-FU | 100 | 75 | 60 | 50 | 40 | 25 | 0 | 0 | 100 |
| roscovitine | 0 | 25 | 40 | 50 | 60 | 75 | 100 | 0 | 100 |

Three different combination strategies were tested:
(A) 5-FU pretreatment wherein cells were treated with 5-FU for 24 hours, then the medium was replaced with roscovitine-containing medium for 48 hours;
(B) Roscovitine pretreatment wherein cells were treated with with roscovitine for 24 hours, then 5-FU for 48 hours;
(C) Cells treated with 5-FU and roscovitine concurrently for 72 hours.

In all three combination strategies, after the 72 hour experimental period, the cells numbers in each well were estimated using the WST1 assay (Roche Applied Science Assay Catalogue No. 1 644 807). Within each plate, cell numbers for each treatment were expressed as a percentage of cells in the control wells (no drug).

Statistical Analysis of Combination Studies

To interpret the combination curves, statistical comparisons were made with each test combination (75:25 roscovitine/5-FU) and the endpoints (100:0-roscovitine and 0:100-5-FU). A statistically significant observation requires that a difference exists between the combination (roscovitine and 5-FU) absorbance value and both endpoint values (roscovitine and 5-FU alone) [Greco et al, The search for synergy; A critical review from a response surface perspective. Pharmacol; Review 47:331-385, 1995; Laska et al, Simple designs and model-free tests for synergy; Biometrics 50:834-841, 1994]. If the majority of (≧3 of 5) of the values are statistically above or below the line (endpoints) then antagonism or synergy is described, respectively. Otherwise, the pattern is more consistent with an additive interaction.

Results

The results for combination strategies (A), (B) and (C) are shown below in Tables 2, 3 & 4, and FIGS. 6, 7 & 8.

TABLE 2

| 5-FU/Roscovitine | | | | | | | |
|---|---|---|---|---|---|---|---|
| Treat- | | | | | | | st. |
| ment | Run 1 | Run 2 | Run 3 | Run 4 | Run 5 | average | dev |
| 1 | 31.89 | 35.51 | 39.15 | 31.68 | 41.56 | 35.96 | 4.38 |
| 2 | 34.49 | 26.94 | 35.7 | 33.7 | 39.92 | 34.15 | 4.69 |
| 3 | 31.13 | 21.52 | 31.87 | 31.95 | 36.02 | 30.50 | 5.37 |
| 4 | 32.43 | 23.52 | 29.27 | 32.87 | 36.62 | 30.94 | 4.90 |
| 5 | 23.75 | 20.57 | 29.73 | 42.36 | 35.2 | 30.32 | 8.77 |
| 6 | 31.13 | 18.95 | 29.14 | 32.97 | 42.37 | 30.91 | 8.39 |
| 7 | 61.93 | 32.94 | 41.29 | 43.09 | 52.85 | 46.42 | 11.19 |

TABLE 3

| 5-FU + | | | | | | | |
|---|---|---|---|---|---|---|---|
| Roscovitine | | | | | | | |
| Treat- | | | | | | | st. |
| ment | Run 1 | Run 2 | Run 3 | Run 4 | Run 5 | average | dev |
| 1 | 30.7 | 19.55 | 30.95 | 19.64 | 41.49 | 28.47 | 9.19 |
| 2 | 27.68 | 14.61 | 21.41 | 14.76 | 31.68 | 22.03 | 7.64 |
| 3 | 23.93 | 13.65 | 15.74 | 11.98 | 23.56 | 17.77 | 5.61 |
| 4 | 26.43 | 6.88 | 13.58 | 9.69 | 19.98 | 15.31 | 7.92 |
| 5 | 29.24 | 3.77 | 13.93 | 9.1 | 20.42 | 15.29 | 9.92 |
| 6 | 32.36 | 7.75 | 12.53 | 9.45 | 15.96 | 15.61 | 9.87 |
| 7 | 22.37 | 14.29 | 19.95 | 16.62 | 30.71 | 20.79 | 6.35 |

TABLE 4

| Roscovitine/5-FU | | | | | | | |
|---|---|---|---|---|---|---|---|
| Treat- | | | | | | | st. |
| ment | Run 1 | Run 2 | Run 3 | Run 4 | Run 5 | average | dev |
| 1 | 57.29 | 40.21 | 53.15 | 56.82 | 36.08 | 48.71 | 9.89 |
| 2 | 62.56 | 44.82 | 43.61 | 51.48 | 31.91 | 46.88 | 11.25 |
| 3 | 56.78 | 41.28 | 38.04 | 43.52 | 30.57 | 42.04 | 9.59 |
| 4 | 57.29 | 37.25 | 32.61 | 50.8 | 29.57 | 41.50 | 11.99 |
| 5 | 49.75 | 38.97 | 34.76 | 54.19 | 31.7 | 41.87 | 9.70 |
| 6 | 41.58 | 36.09 | 35.89 | 52.07 | 43.05 | 41.74 | 6.61 |
| 7 | 17.84 | 22.67 | 31.03 | 40.14 | 45.76 | 31.49 | 11.65 |

TABLE 5

| 5-FU/ | 5-FU/ | Roscovitine/ | | | Roscovitine |
|---|---|---|---|---|---|
| Roscovitine | Roscovitine | 5-FU | 5-FU + Roscovitine | 5-FU (%) | (%) |
| 100/0 | 35.96 | 48.71 | 28.47 | 100 | 0 |
| 75/25 | 34.15 | 46.88 | 22.03 | 75 | 25 |
| 60/40 | 30.5 | 42.04 | 17.77 | 60 | 40 |
| 50/50 | 30.94 | 41.5 | 15.31 | 50 | 50 |
| 40/60 | 30.32 | 41.87 | 15.29 | 40 | 60 |
| 25/75 | 30.91 | 41.74 | 15.61 | 25 | 75 |
| 0/100 | 46.42 | 31.49 | 20.79 | 0 | 100 |

Combination Strategy (A)

The results for pretreatment with 5-FU (shown in Tables 2 & 5, FIG. 6) are indicative of a synergistic interaction between 5-FU and roscovitine.

Combination Strategy (B)

The results for pretreatment with roscovitine (shown in Tables 3 & 5, FIG. 7) are indicative of an additive interaction between 5-FU and roscovitine.

Combination Strategy (C)

The results for concurrent treatment with 5-FU and roscivitine (shown in Tables 3 & 5, FIG. 8) are indicative of a synergistic interaction between 5-FU and roscovitine.

Clonogenic Assay

Preparation of Single Cell Suspensions From Human Tumor Xenografts

Solid human tumor xenografts growing subcutaneously in serial passages in thymus aplastic nude mice (NMRI, Naval Medical Research Institute, USA, nu/nu strain, obtained from breeding facility) were removed under sterile conditions, mechanically disaggregated and subsequently incubated with an enzyme cocktail consisting of collagenase (41 U/ml, Sigma), DNAse I (125 U/ml, Roche), hyaluronidase (100 U/ml, Sigma) and dispase II (1.0 U/ml, Roche) in RPMI 1640-Medium (Life Technologies) at 37° C. for 40 minutes. Cells were passed through sieves of 200 μm and 50 μm mesh size and washed twice with sterile PBS-buffer (Life Technologies). The percentage of viable cells was determined in a Neubauer-hemocytometer using trypan blue exclusion.

Culture Methods

The clonogenic assay was performed in a 24-well format according to a modified two-layer soft agar assay introduced by Hamburger et al [Hamburger, A. W. & S. E. Salmon. 1977; Primary bioassay of human tumor stem cells. Science 197: 461-463]. The bottom layer consisted of 0.2 ml/well of Iscove's Modified Dulbecco's Medium (supplemented with 20% (v/v) fetal calf serum and 0.01% (v/v) gentamicin) and 0.75% (w/v) agar. 5·10⁴ cells were added to 0.2 ml of the same culture medium supplemented with 0.4% (w/v) agar and plated in 24-multiwell dishes onto the bottom layer. Cytostatic drugs were applied by continuous exposure (drug overlay) in 0.2 ml culture medium 24 hours after seeding in the cells. Every dish included six control wells and drug-treated groups in triplicate at 6 concentrations. In combination studies, roscovitine and the standard cytotoxic agent were applied simultaneously, 3-fold concentrated to end up in the respective test concentration which is reached by diffusion through the assay layers. The cytotoxic agent was tested in 6 dilutions and roscovitine was added in 2 constant concentrations to the respective dilutions. Cultures were incubated at 37° C. and 7.5% CO₂ in a humidified atmosphere for 8-20 days and monitored closely for colony growth using an inverted microscope. Within this period, in vitro tumor growth led to the formation of colonies with a diameter of >50 μm. At the time of maximum colony formation, counts were performed with an automatic image analysis system (OMNICON FAS IV, Biosys GmbH). 24 hours prior to evaluation, vital colonies were stained with a sterile aqueous solution of 2-(4-iodophenyl)-3-(4-nitrophenyl)-5-phenyltetrazolium chloride (1 mg/ml, 100 μl/well) (Alley, M. C., Uhi, C. B. & M. M. Lieber, 1982. Improved detection of drug cytotoxicity in the soft agar colony formation assay through use of a metabolizable tetrazolium salt. Life Sci. 31: 3071-3078).

An assay was considered fully evaluable, if the following quality control criteria were fulfilled:
Mean number of colonies in the control group wells of 24-multiwell plates≧20 colonies with a colony diameter of >50 μm;
Coefficient of variation in the control group≦50%;
The positive reference compound 5-fluorouracil (5-FU) (at the toxic dose of 1000 μg/ml) must effect a colony survival of <20% of the controls; or
Initial plate counts on day 0 or 2<20% of the final control group count.
   Data Evaluation

Drug effects were expressed in terms of the percentage of survival, obtained by comparison of the mean number of colonies in the treated plates with the mean colony count of the untreated controls (relative colony count expressed by the test-versus-control-group value, T/C-value [%]):

$$\frac{T}{C} = \frac{colony {count}_{treated group}}{colony {count}_{control group}} \cdot 100 [ \% \} .$$

In combination studies, T/C-values obtained for the combination at each drug dosage were compared to the T/C-values obtained with roscivitine or the standard agent at the respective concentration alone. A benefit of combination was obtained when T/C-values from combinations were significantly lower than the T/C-values of the respective monotherapy. IC₅₀ and IC₇₀ values, being the drug concentration necessary to inhibit colony formation by 50% (T/C=50%) and 70% (T/C=30%) respectively, were determined by plotting compound concentration versus relative colony count.

The results are shown in FIG. 9. When the human mammary cancer xenograft MAXF 857 was treated with 10 μM roscovitine alone this gave a relative colony count of 77%. When this cell line was treated with the combination of roscovitine and 5FU the relative colony count was approximately 45% at concentrations of 5FU that had no effect on cell growth when treated alone. Thus the reduction from 77% to 45% suggests significant synergy in this concurrent treatment.

By way of summary, the efficacy of combination treatment using roscovitine and 5-FU has been demonstrated using a variety of experimental methods, including cell cycle analysis, cell proliferation studies, and studies on the induction of apoptosis. It is noteworthy that the results of studies using MCF7 cells indicate the presence of a synergistic effect where 5-FU and roscovitine are administered concurrently, and also for pretreatment with 5-FU. Studies on the induction of apoptosis as measured by the activated caspase-3 assay indicate the presence of a synergistic effect for pretreatment with roscovitine.

Thus, the experimental results outlined above provide evidence of synergism where 5-FU and roscovitine are administered concurrently, and for sequential administration in any order, i.e. where 5-FU is administered prior to roscovitine, and where roscovitine is administered prior to 5-FU.

Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

1. A combination comprising a CDK inhibitor and 5-FU, or a prodrug thereof.

2. A combination according to claim 1 wherein the CDK inhibitor is an inhibitor of CDK2 or CDK4.

3. A combination according to claim 1 or claim 2 wherein the CDK inhibitor is selected from rosovitine, purvalanol A, purvalanol B and olomoucine.

4. A combination according to any preceding claim wherein the CDK inhibitor is roscovitine.

5. A combination according to any preceding claim wherein the prodrug of 5-FU is capecitabine.

6. A pharmaceutical composition comprising a combination according to any preceding claim and a pharmaceutically acceptable carrier, diluent or excipient.

7. Use of a combination according to any one of claims 1 to 5 in the preparation of a medicament for the treatment of a proliferative disorder.

8. Use according to claim 7 wherein the proliferative disorder is cancer.

9. Use according to claim 8 wherein the cancer is breast cancer.

10. A pharmaceutical product comprising a CDK inhibitor and 5-FU, or a prodrug thereof, as a combined preparation for simultaneous, sequential or separate use in therapy.

11. A pharmaceutical product according to claim 10 for separate or sequential use in therapy, wherein the 5-FU, or prodrug thereof, and CDK inhibitor are administered sequentially.

12. A pharmaceutical product according to claim 10 or claim 11 wherein the CDK inhibitor is an inhibitor of CDK2 or CDK4.

13. A pharmaceutical product according to any one of claims 10 to 12 wherein the CDK inhibitor is selected from rosovitine, purvalanol A, purvalanol B and olomoucine.

14. A pharmaceutical product according to any one of claims 10 to 13 wherein the CDK inhibitor is roscovitine.

15. A pharmaceutical product according to any one of claims 10 to 14 wherein the prodrug of 5-FU is capecitabine.

16. A pharmaceutical product according to any one of claims 10 to 15 in the form of a pharmaceutical composition comprising a pharmaceutically acceptable carrier, diluent or excipient.

17. A pharmaceutical product according to any one of claims 10 to 16 for use in the treatment of a proliferative disorder.

18. A pharmaceutical product according to claim 17 wherein the proliferative disorder is cancer.

19. A pharmaceutical product according to claim 18 wherein the proliferative disorder is breast cancer.

20. A method of treating a proliferative disorder, said method comprising administering to a subject, simultaneously, sequentially or separately, 5-FU or a prodrug thereof, and a CDK inhibitor.

21. A method according to claim 20 which comprises administering said CDK inhibitor to a subject prior to sequentially or separately administering 5-FU, or a prodrug thereof, to said subject.

22. A method according to claim 20 which comprises administering 5-FU, or a prodrug thereof, to a subject prior to sequentially or separately administering a CDK inhibitor to said subject.

23. A method according to any one of claims 20 to 22 wherein the CDK inhibitor is an inhibitor of CDK2 or CDK4.

24. A method according to claim 23 wherein the CDK inhibitor is selected from rosovitine, purvalanol A, purvalanol B and olomoucine.

25. A method according to claim 24 wherein the CDK inhibitor is roscovitine.

26. A method according to any one of claims 20 to 25 wherein the prodrug of 5-FU is capecitabine.

27. A method according to any one of claims 20 to 26 wherein the CDK inhibitor and 5-FU, or prodrug thereof, are each administered in a therapeutically effective amount with respect to the individual components.

28. A method according to any one of claims 20 to 26 wherein the CDK inhibitor and 5-FU, or prodrug thereof, are each administered in a subtherapeutic amount with respect to the individual components.

29. A method according to any one of claims 20 to 28 wherein the proliferative disorder is cancer.

30. A method according to any one of claims 20 to 29 wherein the proliferative disorder is breast cancer.

31. Use of a CDK inhibitor in the preparation of a medicament for the treatment of a proliferative disorder, wherein said treatment comprises administering to a subject simultaneously, sequentially or separately 5-FU, or a prodrug thereof, and a CDK inhibitor.

32. Use of a CDK inhibitor and 5-FU, or a prodrug thereof, in the preparation of a medicament for treating a proliferative disorder.

33. Use of a CDK inhibitor in the preparation of a medicament for the treatment of a proliferative disorder, wherein said medicament is for use in combination therapy with 5-FU, or a prodrug thereof.

34. Use of 5-FU, or a prodrug thereof, in the preparation of a medicament for the treatment of a proliferative disorder, wherein said medicament is for use in combination therapy with a CDK inhibitor.
